(19) United States
(12) Patent Application Publication
(10) Pub. No.: US 2008/0026074 A1
(43) Pub. Date: Jan. 31, 2008

(54) Method of short term skin wrinkles correction using strontium divalent ion

(76) Inventors: Hannah Naomi Sivak, Gilbert, AZ (US); Mary Hunter, El Paso, TX (US)

Correspondence Address: Hannah Sivak, 1213 W. Seascape Dr., Gilbert, AZ, 85233, US

(21) Appl. No.: 11/494,776

(22) Filed: Jul. 27, 2006

(51) Int. Cl. A61K 33/24; A61K 38/05; A61K 31/724; A61K 36/73; A61K 31/665; A61K 31/192

(52) U.S. Cl. 424/617; 424/765; 514/18; 514/58; 514/100; 514/568

(57) ABSTRACT

A method for the treatment of skin damage using strontium divalent ion in a dermatologically acceptable carrier is provided, usually an essential oil distillate. The compositions are applied topically to improve appearance of skin damaged by sun or by natural aging. The compositions may additionally include antioxidant molecules such as vitamin E, ferulic acid and vitamin C.

### FIELD OF THE INVENTION

The present invention generally relates to compositions and methods for temporary improving the appearance of aged skin and more particularly to compositions including strontium salts and essential oil distillates and to methods of using and forming the composition.

### BACKGROUND OF THE INVENTION

Aging of the population and long term exposure to the sun in previous decades when tanning was fashionable, has resulted in a large percentage of the population having photodamaged skin. Because of the longer life expectancy and changes in life style, the skin changes resulting from damage by UV are no longer acceptable to many adults, especially those from the so called “baby boom generation”.

The anatomy and biochemistry of wrinkled skin is complicated and includes permanent changes in the structure of the dermis and epidermis and changes in the structure of the macromolecules involved in maintenance of skin elasticity and thickness.

Because of the complexity of mechanisms involved in the development of wrinkles, actual improvement in the anatomy and biochemistry of wrinkled skin is a long process and can only be improved to some extent. For this reason, short term cosmetic treatments are always sought that will smooth the skin for a few weeks and even a few hours.

There are many treatments purported to improve the appearance of aging skin, but only one of them, injections of botulinum toxin, has a clear effect on wrinkles by inhibiting the capacity of the facial muscles to contract. Botulinum toxin works by binding to the neuronal cell membrane. The light polypeptide chain of the two-chain botulinum toxin cleaves specific sites on the SNARE proteins, preventing complete assembly of the synaptic fusion complex and thereby blocking acetylcholine release. Without acetylcholine release, the muscle is unable to contract and the wrinkle is smoothed out.

Use of botulinum toxin for wrinkles is only suitable for a minority because it requires injections administered every few months by medical professionals, making it an expensive proposition. Besides, the method has the potential for causing accidental permanent paralysis of face muscles.

Other treatments purported to smooth out wrinkles consist in the application of creams containing peptides designed to have some homology to the SNARE complex of SNAP-25, thus interfering with muscle contraction. These treatments require long term use and seem to be of doubtful efficacy.

Accordingly, compositions including materials to help improve the skin appearance, decreasing the depth of wrinkles are desired.

### DETAILED DESCRIPTION

The present invention provides methods of improving the appearance of sun damaged skin and/or aged skin. More particularly, the invention provides methods of treating skin using compositions that include strontium divalent ions.

The entry of calcium into cells via voltage dependent channels regulates muscle contraction and neuronal discharge.

Some divalent inorganic ions, such as barium and strontium, mimic the ability of calcium to pass through the voltage dependent calcium channels. Strontium (Sr) is located below Calcium in the Periodic Table of the Elements. Sr⁺⁺ is a divalent cation that may also compete with Ca⁺⁺ for binding to some receptors. This capacity of strontium ions maybe responsible for the analgesic effect of strontium, an effect taken advantage of in dentifrices, alleviation of skin sensory irritation and, taken orally, for arthritic pain. Calcium is thought to play a role in the pain process by regulating the release of the chemicals that nerve cells use to communicate, and strontium would compete with calcium, effectively hindering entrance of calcium into the nerve cell.

Strontium is taken orally to help with osteoporosis. This particular use has led to extensive toxicity studies and it has been found that even for long term use, toxicity is not a problem.

We have found that application of strontium ion in a suitable carrier, e.g. a distillate of orange flower petals (neroli floral water), within minutes of application changes the texture of photoaged skin, resulting in a more youthful complexion. The epidermal ridges are less pronounced, an effect that may be related to relaxation of erector muscles.

Application of a lotion containing 6 grams of strontium ion per 100 mL, using as diluent distillate of orange petals and distilled water in equal volume, smoothed out the skin and increased skin turgidity within a few minutes. This effect lasted for about 4 hours.

This smoothing effect could be observed even when the strontium solution was applied over a layer of powder make-up.

The objective of this invention is to provide a method and composition for a cosmetic treatment that improves the texture of skin, decreasing the appearance of wrinkles and increasing the turgidity based upon the topical application to exposed or affected skin areas of at least one active agent, in association with a dermatologically acceptable carrier or vehicle. This invention is based upon the finding that strontium divalent ions in a suitable vehicle can have a smoothing effect upon skin.

These and other objectives are accomplished by the present invention, which provides methods and compositions for the cosmetic treatment of skin, by applying topically to the exposed or affected skin areas an effective amount of strontium divalent ion, preferably in a dermatologically acceptable carrier.

The present invention also provides methods and compositions for the improved appearance of skin, by applying to it an effective amount of strontium salt or salts, preferably in an acceptable carrier.

As used herein, the term “strontium ion” encompasses any strontium salt that is capable of exerting a similar action on skin.

Strontium chloride hexahydrate is a preferred salt because of the lack of negative effects of the counterion, chloride, on the skin.

Many embodiments incorporate at least one other active ingredient with the strontium salt. These include natural or synthetic antioxidant molecules such as reduced glutathione, vitamin E, ascorbic acid, and/or lycopene. Other desirable ingredients are proteins capable of alleviating oxidative stress such as catalase, thioredoxin and/or superoxide dismutase.

In the preferred practice of the invention, strontium chloride hexahydrate is applied in admixture with a dermatologically acceptable carrier or vehicle (e.g., as a solution, lotion, cream, ointment, serum) so as to facilitate topical application and, in some cases, provide additional therapeutic effects as might be brought about by moisturizing the affected skin areas. As noted, other ingredients are advantageously included in the compositions.

The amount of strontium ion necessary to bring about an improvement is skin appearance is not fixed, and is dependent upon the source, purity and activity of the salt employed, the amount and type of any additional ingredients used, particularly those that appear to exhibit synergistic effects, the skin type of the user, and, where present, the severity and extent of skin damage.

In one embodiment, the composition contains from about 0.1% to about 10% (weight per volume), preferably from more than 1% to about 6% strontium ion.

While the carrier for strontium ion can be very simple (such as saline solution), it is generally preferred that the carrier be a composition that will facilitate topical application, and particularly one which will form a film or layer on the skin to which it is applied so as to localize the active ingredient. Many such compositions are known in the art, and can take the form of lotions, creams, gels, etc. Typical compositions include lotions containing water and/or alcohols and emollients such as natural oils and waxes, silicone oils, hyaluronic acid, glyceride derivatives, fatty acids or fatty acid esters or alcohols or alcohol ethers, lanolin and derivatives, polyhydric alcohols or esters, wax esters, sterols, phospholipids and the like, and generally also emulsifiers (nonionic, cationic or anionic), although some of the emollients inherently possess emulsifying properties. These same general ingredients can be formulated into a cream rather than a lotion, or into gels, or into solid sticks by utilization of different proportions of the ingredients and/or by inclusion of thickening agents such as gums or other forms of hydrophilic colloids. Such compositions are referred to herein as dermatologically-acceptable carriers.

Many preferred embodiments of this invention contain at least one or two, and sometimes several, other active ingredients in addition to strontium, provided that the ingredients do not interfere with the action of strontium, such as calcium.

Reduced glutathione, magnesium ascorbyl phosphate, lycopene, vitamin E, etc. may also be added to the epidermal growth factor composition, alone or in combination with other ingredients in some embodiments.

In terms of a possible explanation for the effectiveness of the active ingredients in the prevention or treatment of damage to the skin, it is noted that strontium may be acting through competition with calcium for receptors or channels.

Having described the invention with reference to particular compositions, theories of effectiveness, it will be apparent to those of skill in the art that it is not intended that the invention be limited by such illustrative embodiments or mechanisms, and that modifications can be made without departing from the scope or spirit of the invention, as defined by the appended claims. It is intended that all modifications and variations be included within the scope of the invention. The claims are meant to cover the claimed components and steps in any sequence which is effective to meet the objectives there intended, unless the context specifically indicates the contrary.

We claim:

1. A method for the short-term cosmetic treatment of photoaged skin which consists of applying a composition containing strontium divalent ion, or a strontium salt in a dermatologically acceptable carrier to the affected skin area.

2. A method for the short-term cosmetic treatment of photoaged skin which consists of applying a composition containing neroli or rose petals distillate or distilled water with essential oil in conjunction with strontium divalent ion.

3. A method in accordance with claims 1 or 2, wherein said compositions further comprise one or more additional ingredients selected from the group consisting of magnesium ascorbyl phosphate, ferulic acid, beta cyclodextrins, reduced glutathione.
